(19) United States
(12) Patent Application Publication
(10) Pub. No.: US 2010/0119411 A1
(43) Pub. Date: May 13, 2010

(54) APPARATUS AND METHOD TO DELIVER A STERILE, FILLED SYRINGE TO A USER

(76) Inventors: Ashok V. Joshi, Salt Lake City, UT (US); James Steppan, Park City, UT (US)

Correspondence Address: CERAMATEC, INC., 2425 SOUTH 900 WEST, SALT LAKE CITY, UT, 84119, US

(21) Appl. No.: 12/268,577

(22) Filed: Nov. 11, 2008

(51) Int. Cl. A61L 2/20; B01J 19/08; C25C 7/04

(52) U.S. Cl. 422/29; 422/28; 422/186.07; 204/252

(57) ABSTRACT

A stand-alone apparatus to deliver a sterile, filled syringe to a user. The syringe dispenser includes a controller to accept input from the user and to convert the input into an electrical control signal. The syringe dispenser also includes an ozone generator coupled to the controller. The ozone generator generates ozone on demand according to the input from the user. The user may input a parameter for a concentration and/or a volume of ozone. Additionally, the syringe dispenser includes a syringe preparation station coupled to the ozone generator. The syringe preparation station sterilizes the syringe with a first amount of the ozone and fills the syringe with a second amount of the ozone.

### BACKGROUND

Use of sterile syringes is a standard industry practice in the medical field. Prior to filling a syringe with a medicinal or therapeutic agent of choice, the filled syringe is sterilized to remove potential contaminants. In many cases, the syringes are packaged in sterilized conditions. Additionally, some syringes may come prefilled with therapeutic agents and delivered in a sterilized condition within hermetically sealed pouches which are delivered to treatment facilities. However, pre-filled sterilized syringes have the limitation of coming in pre-determined concentrations and amounts. Additionally, some therapeutic agents such as ozone are not suitable for use in prefilled syringes due to the shelf life of the agents.

Ozone as a therapeutic agent of choice may be used as a curative for herniated disks, rheumatoid arthritis, and osteoarthritis. Ozone may also be used therapeutically to treat inflammation such as bursitis of a knee, shoulder, or hip. Additionally, there may be veterinary applications of ozone to animals suffering from disc and degenerative diseases.

Lavage of a surgical space prior to placement of a permanent surgical implant such as a hip or knee prosthesis, or pacemaker or treatment of an infected joint can be facilitated by the use of medical ozone as a sterilizing substance. Similarly, a colostomy stoma can be created such that the adhesive disk is infused with ozone to aid in healing and inhibit infection. The post surgical recovery from sternotomy after cardiac surgery is often complicated by wound infection. Placement of a resorbable catheter in the wound that could be irrigated with ozone would aid healing. Indeed, many wounds could have a resorbable multisided hole catheter placed in it to allow ozone to be injected through it. This would have anti-infective, analgesic, and wound healing properties. This would shorten recovery time and decrease complication rates after surgery. Ozone administration can be performed by directly delivering an ozone gas mixture, or a liquid or gel that contains ozone, to the treatment site.

Ozone could be applied to a site of high probability of infection such an abdominal incision/wound after appendectomy, or urgent colectomy with colostomy or after percutaneous endoscopic cholecystectomy. Endoscopic procedural infusion of ozone and trans catheter infusion of ozone can be used to inhibit the complications due to endoscopic medical intervention or image guided or non-image guided catheter based intervention, for example, in endoscopic evaluation of the pancreatic duct. Dental injection of ozone may also augment the preparation and repair of dental cavities, and aid in reduction of root canal inflammation or periodontal disease. The therapeutic and medicinal applications of ozone are being continually researched.

In some applications, ozone is also used for sterilization purposes. Unless bound by other molecular couplings, ozone may break down to dioxygen within 20 to 30 minutes or so at atmospheric pressure. Ozone is highly reactive with many substances not desired in the human body including yeast, mold, water borne parasites and harmful bacteria. Ozone is therefore used as a sterilizing agent for medical equipment in hospitals and to sterilize food and laundry in care facilities, as well as in many other environments and applications.

As the success of ozone gas therapy continues to gain recognition in medical and therapeutic applications, there is a lack of conventional methods and products to effectively implement ozone generation and ozone sterilization.

### SUMMARY

According to described embodiments, a syringe dispenser to deliver a sterile, filled syringe to a user is disclosed. The syringe dispenser includes a controller to accept input from the user and to convert the input into an electrical control signal used primarily within the apparatus. The syringe dispenser also includes an ozone generator coupled to the controller. The ozone generator generates ozone on demand according to the input from the user. For instance, the user may input a parameter for a concentration and/or a volume of ozone. Additionally, a syringe preparation station is coupled to the ozone generator. The syringe preparation station sterilizes the syringe with a first amount of the ozone and fills the syringe with a second amount of the ozone.

In another embodiment, a syringe dispenser to deliver a sterile, filled syringe to a user may additionally include an ozone sensor coupled to the ozone generator and to the controller. The ozone sensor senses a characteristic of the ozone. The syringe dispenser also may include a scrubber to receive excess ozone from the ozone generator and dispose of the excess ozone. Other embodiments of the syringe dispenser are also described.

Embodiments for a method of autonomously sterilizing and filling a syringe are also described. The method includes automatically moving the syringe from a syringe repository to a sterilization station and generating ozone on demand from an oxygen source. The generated ozone is used both to sterilize the syringe and to fill the syringe. Other embodiments of the method are also described herein.

Other aspects and advantages of embodiments of the present invention will become apparent from the following detailed description, taken in conjunction with the accompanying drawings, illustrated by way of example of the principles of the invention.

### BRIEF DESCRIPTION OF THE DRAWINGS

FIG. 1 depicts a schematic diagram of one embodiment of a syringe dispenser to deliver a sterile, filled syringe to a user.

FIG. 2 depicts a block diagram of one embodiment of a syringe dispensing system to deliver a sterile, filled syringe to a user.

FIG. 3 depicts one embodiment of a process flow diagram for generating ozone from water in the syringe dispenser of FIG. 2.

FIG. 4 depicts one embodiment of a process flow diagram for generating ozone from air in the syringe dispenser of FIG. 2.

FIG. 5 depicts one embodiment of a process flow diagram for generating ozone from steam in the syringe dispenser of FIG. 2.

FIG. 6 depicts a flow chart diagram of an embodiment of a method for autonomously sterilizing and filling a syringe.

FIG. 7 depicts a flow chart diagram of an embodiment of a method for generating ozone according to user input.

Throughout the description, similar reference numbers may be used to identify similar elements.

### DETAILED DESCRIPTION

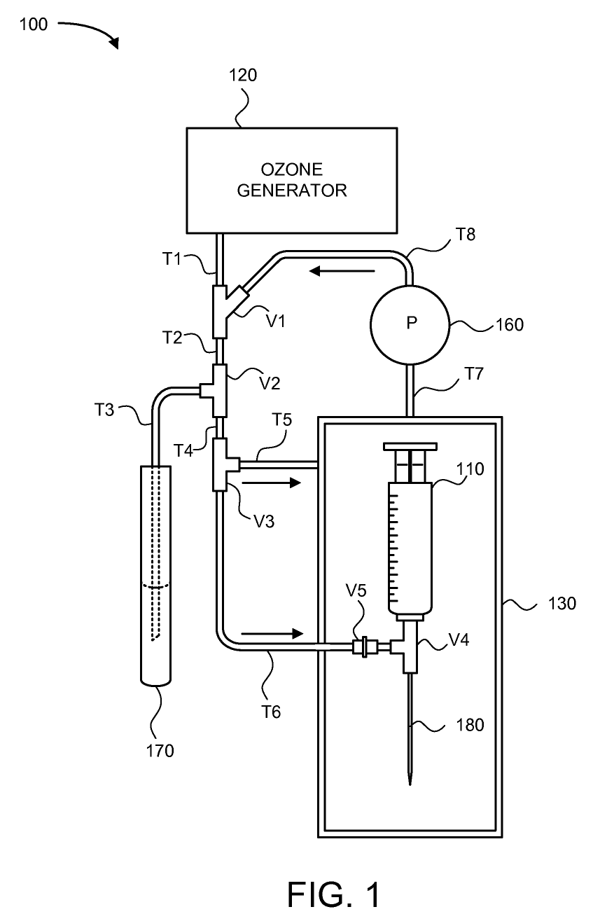

FIG. 1 depicts a schematic diagram of one embodiment of a syringe dispenser 100 to deliver a sterile, filled syringe 110 to a user. The illustrated syringe dispenser 100 includes a syringe 110, an ozone generator 120, a syringe preparation station 130, a pump 160, and a scrubber 170. The various components of the syringe dispenser 100 are coupled together by several channels of tubing, designated as T1-T8, as well as several valves, designated as V1-V5. Although the syringe dispenser is illustrated with several components and interconnections, other embodiments may include fewer or more components to implement more or less functionality. Additionally, some embodiments may implement different connection paths through the same or different channels of tubing and valves, or other connecters.

In general, the ozone generator 120 generates ozone on demand according to input from a user. The syringe preparation station 130 contains the syringe 110 and is connected to the ozone generator 120. The syringe preparation station 130 is configured to sterilize the syringe 110 with a first amount of the generated ozone and to fill the syringe 110 with a second amount of the generated ozone. The pump 160 may activate to facilitate moving the ozone from the ozone generator 120 to the syringe preparation station 130 and/or from the syringe preparation station 130 to a scrubber or ozone destruction unit 170. The scrubber 170 decomposes the excess ozone evacuated from the syringe preparation station 130. Additionally, the scrubber 170 may receive and destroy the excess ozone from the syringe 110. In some embodiments, the scrubber 170 is a charcoal, potassium iodide (KI), magnesium dioxide (MnO₂), and/or copper oxide (CuO) filter. In other embodiments, the scrubber 170 is a thermal-type ozone destruct unit.

Referring more specifically to FIG. 1, the ozone generator 120 can be based on any known ozone generator. The ozone generator 120 connects to the scrubber 170 and the syringe preparation station 130 via the flexible tubing and valves for selectively directing the flow of gas therebetween. More specifically, a first channel of tubing T1 connects the generator 120 to a three-way valve V1. A second channel of tubing T2 connects the valve V1 to another three-way valve V2. The valve V2 is further connected to the scrubber 170 through another channel of tubing T3. A third three-way valve V3 is connected by another channel of tubing T4 to the second three-way valve V2. The valve V3 is also connected to the syringe preparation station 130 by another channel of tubing T5 and to the syringe 110 within the syringe preparation station 130 by a separate channel of tubing T6. The tubing is made of any suitable material, such as silicone or Teflon of the known medical types, and has a diameter and wall thickness to withstand the pressure of ozone gas being carried therethrough. The valves, also known as stopcocks, are also of the known medical type and have fittings complementary to the various portions of tubing.

In the illustrated embodiment, the syringe 110 includes a three-way valve V4 and a needle 180. The syringe 110 may be made of polyethylene, or another similar material, to resist the corrosive effect of ozone. The three-way valve V4 is releasably connected directly to the valve V5, providing a selective pathway between the syringe 110 and the ozone generator 120 and/or the scrubber 170.

The valves V1 and V2 have a first position to allow the ozone generator 120 to transfer ozone to the syringe preparation station 130 through the tubing channels T1, T2, and T3, as well as the valve V3. The valve V3 has a first position which allows the ozone to flow from the tubing channel T4 into the syringe preparation station 130 via the tubing channel T5. By directing the ozone into the syringe preparation station 130 via the tubing channel T5, the syringe 110 within the syringe preparation station 130 may be sterilized as the ozone collects within the syringe preparation station 130. Further details about sterilizing the syringe 110 in this manner are provided below.

The valve V3 has a second position which allows the ozone to flow from the tubing channel T4 into the syringe preparation station 130 via the tubing channel T6. By directing the ozone into the syringe preparation station 130 via the tubing channel T6, the syringe 110 may be filled with a volume of the ozone from the ozone generator 120. More specifically, the ozone is directed through the valve V5, which may be used to connect to the syringe 110, and the ozone is directed into the valve V4 to fill the syringe 110. Further details about filling the syringe 110 with the ozone in this manner are provided below.

Thus, by toggling the valve V3 when the ozone generator 120 is on, the syringe 110 is sterilized within the syringe preparation station 130 and the syringe 110 is filled with ozone. In other embodiments, the valve V3 may be omitted and the valve V4 may be configured to fill the syringe 110 and to release additional ozone into the syringe preparation station 130 for sterilizing the syringe 110. Therefore toggling the valve V4 will allow sterilizing the syringe 110 with a first amount of ozone from the ozone generator 120 and filling the syringe 110 with a second amount of ozone from the ozone generator 120. Other embodiments may implement similar functionality using fewer or more valves and/or tubing channels, or configurations thereof.

In some embodiments, excess ozone from the syringe preparation station 130 is evacuated to the scrubber 170 through the tubing channels T7, T8, T2, and T3. In some embodiments, the pump 160 creates a vacuum condition within the syringe preparation station 130, and the valves V1 and V2 direct the evacuated ozone into the scrubber 170. In particular, the valve V1 has a second position which facilitates the flow of ozone from the pump 160 to the valve V2. Therefore, when the valves V1 and V2 are in the second position, the pump 160 may evacuate excess ozone from the syringe preparation station 130 into the scrubber 170. It should be noted that the pump 160 may also be placed in other channels between valves, or multiple pumps may be placed throughout the syringe dispenser 100 to facilitate ozone flow and/or evacuation. In some embodiments, the syringe dispenser 100 may operate without a pump. In some embodiments, the ozone generator 120 includes an internal pump.

Additionally, excess ozone from the ozone generator 120 may be directed to the scrubber 170 via the tubing channels T1, T2, and T3, as well as the valves V1 and V2. In particular, the valve V2 has a second position which blocks flow to the tubing channel T4 and the valve V3. As an additional safeguard, the valve V5 has a closed position to effectively cap the tubing channel T6, preventing flow from the tubing channel T6 to the syringe 110. Therefore, when valve V2 is in the second position and valve V1 is in the first position, any ozone generated by ozone generator 120 is captured by scrubber 170.

Any excess ozone still present in the tubing may also be captured by the scrubber 170 and thereby reduce and/or substantially eliminate the unwanted escape of ozone into the atmosphere where it may harm the operator or other individuals proximal to apparatus 100. Once generator 120 is turned “off” after filling and sterilizing syringe 110 as described above, valves V1, and V2 are each moved from their respective first position to their respective second position, and valve V3 is toggled to open up channels 140 and 150

The valve V4 also has a second position which prevents backflow from the syringe 110 to the valve V5 (or the open fitting on the valve V4 that connects to the valve V5). Additionally, the valve V4 may prevent the flow of ozone out of the needle 180 of the syringe 110, in the absence of sufficient pressure or mechanical control of the valve V4. Thus, once the syringe 110 is filled with ozone, the valve V4 is placed in the second position to retain the ozone within the syringe 110 even after the syringe 110 is disconnected from the valve V5 and dispensed to a user.

Once the syringe 110 is charged, or filled, with ozone, the syringe 110 can be dispensed from the syringe dispenser 100 so that the syringe 110 can be used to administer ozone, for example, to a target area of a patient. Thus, the valve V4 also has a third position that places the syringe 110 in communication with needle 180 for discharging the ozone from the syringe 180.

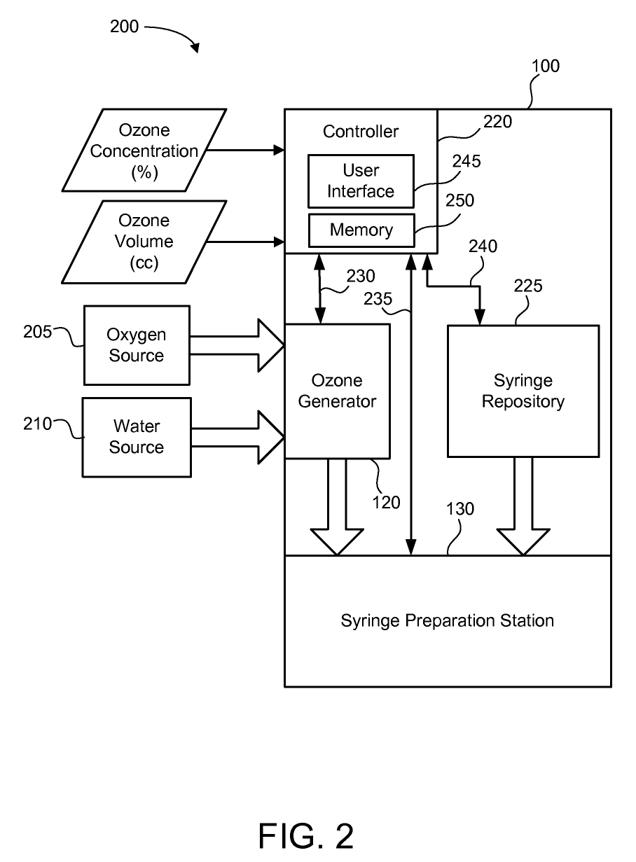

FIG. 2 depicts a block diagram of one embodiment of a syringe dispensing system 200 to deliver a sterile, filled syringe 110 to a user. The illustrated syringe dispensing system includes the syringe dispenser 100 of FIG. 1, as well as an oxygen source 205 and a water source 210. In general, the syringe dispenser 100 receives user input (e.g., ozone concentration (%), ozone volume (cc), etc.) and oxygen, air, or water in order to generate the ozone. The illustrated syringe dispenser includes the ozone generator 120, the syringe preparation module 130, a controller 220, and a syringe repository 225.

The controller 220 is connected to the ozone generator 120 via a first communication channel 230, to the syringe preparation station 130 via a second communication channel 235, and to the syringe repository via a third communication channel 240. In one embodiment, the controller 220 accepts the user input from a user and converts the user input into at least one electrical control signal transferred over communication channels 230, 235, and 240. The depicted controller 220 includes a user interface 245 and a memory device 250. The user may input a parameter such as a concentration of ozone or a volume of ozone into the user interface 245, which stores the user input in the memory device 250. The user may input the concentration and volume, or other parameters, through the user interface 245 such as a keypad to the controller 120 or through other methods such as voice recognition or document scanning. Alternatively, the parameters (e.g., concentration and volume) may be stored in a memory 250 from source other than the user interface 245. Based on the stored parameters or the user input directly, the controller 220 then may send electrical signal(s) to the ozone generator 120 to control the generation of a volume and concentration of ozone to be generated. Other parameters to characterize the ozone may also be input by the user via the user interface 245 or stored in the memory device 250.

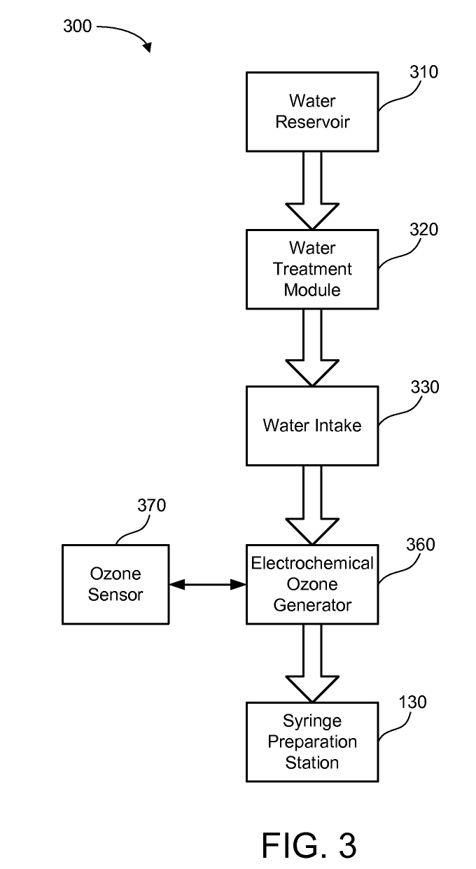

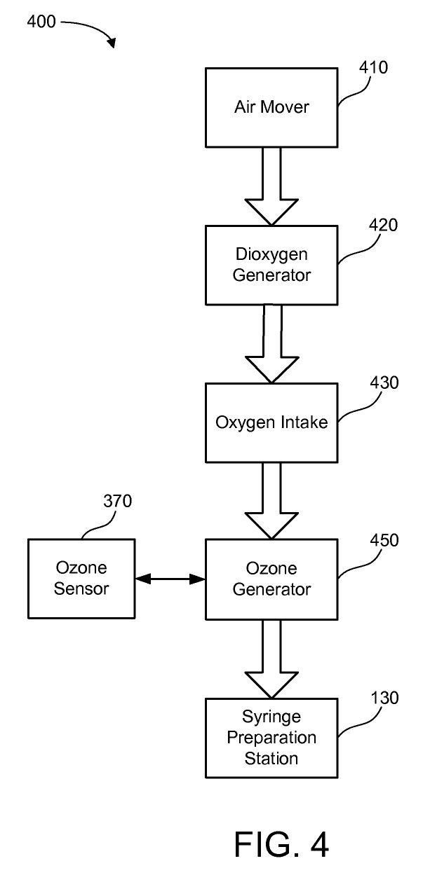

In one embodiment, the ozone generator uses oxygen from the oxygen source 205 and/or water from the water source 210 to generate the ozone. Additional details about generating ozone from air are shown in FIG. 4 and described below. Additional details about generating ozone from water are shown in FIG. 3 and described below.

Generally, oxygen constitutes about 88.8% of the mass of water and about 20.9% of the volume of air. Therefore, a larger amount of ozone may be generated from a volume of water than from a comparable volume of air. In one embodiment, the water source 210 provides sterile, deionized water to facilitate ozone generation. In another embodiment, the water source 210 provides tap or soft water into the ozone generator 120.

In one embodiment, the controller 220 also controls the valves depicted in FIG. 1 and other mechanical systems (not shown) within the syringe dispenser 100. For example, the controller 220 may control a mechanical element to move the syringe 110 within the dispenser 100 from the syringe repository 225 to the syringe preparation station 130. The syringe repository 225 may hold one or several unfilled syringes prior to sterilization and filling of the stored syringes. Additionally, in some embodiments, the syringe dispenser 100 may include a syringe dispenser (not shown) coupled to the syringe repository 225 to provide an unfilled syringe to a user prior to sterilization and/or filling. In some embodiments, the syringe dispenser 100 includes a separate syringe repository (not shown) which stores used syringes for disposal. In some embodiments, the used syringes may be sterilized prior to storage and batch disposal. Further, in some applications, it may be useful to sterilize used syringes that have been used previously, either for sanitation and disposal or for reuse (in jurisdictions with applicable safety standards).

The syringe preparation station 130 may include a dispensary to vend the sterile, filled syringe 110 to a user. The dispensary may be part of the syringe preparation station 130. Alternatively, the dispensary may be separate from the syringe preparation station 130.

FIG. 3 depicts one embodiment of a process flow diagram 300 for generating ozone from water in the syringe dispenser 100 of FIG. 2. In one embodiment, a water reservoir 310 holds water for a water treatment module 320. The water treatment module 320 treats the water using deionization and other conventional treatment methods to supply deionized water to a water intake 330. The water intake 330 delivers the water from the water treatment module 320 to an electrochemical ozone generator 360. The electrochemical ozone generator 360 generates ozone from the deionized water using conventional electrochemical processes.

In one embodiment, an ozone sensor 370 monitors the ozone generated by the electrochemical ozone generator 360. In some embodiments, the ozone sensor 370 may communicate with the controller 220 to adjust the concentration, volume, or another characteristic of the generated ozone. Alternatively, the controller 220 may communicate a feedback signal directly to the electrochemical ozone generator 360 in order to adjust a characteristic of the generated ozone, according to the user input. The electrochemical ozone generator 360 sends the generated ozone to the syringe preparation station 130 for use in sterilizing and/or filling the syringe 110.

FIG. 4 depicts one embodiment of a process flow diagram 400 for generating ozone from air in the syringe dispenser 100 of FIG. 2. In one embodiment, an air mover 410 brings ambient air to a dioxygen generator 420. The air mover 410 may be a fan, a negative pressure induction device, or another type of device which facilitates the flow of air toward or into the dioxygen generator 420. In another embodiment, the air mover 410 may supply treated air from a tank (not shown) or other source to the dioxygen generator 420. The dioxygen generator 420 generates dioxygen on demand from the ambient air. An oxygen intake 430 moves the dioxygen from the dioxygen generator 420 into an ozone generator 450 to produce ozone of substantial purity, as monitored by the ozone sensor 370. The ozone generator 450 sends the generated ozone to the syringe preparation station 130. In an alternative embodiment, the air mover 410 and the dioxygen generator 420 may be omitted, and oxygen from a tank (not shown) or other source may be directly supplied to the oxygen intake 430. In another embodiment, ambient air may be supplied directly to the oxygen intake 430.

There are many types of ozone generators 450 which may be used to generate the ozone from the dioxygen. For example, ozone may be made by applying ultra violet (UV) energy or electrical discharge energy to either pure oxygen, or more commonly just plain air. Therefore, the ozone generator 450 may be a corona discharge generator or a UV light. In such embodiments, the ozone generated from the corona discharge generator or the UV light is delivered to the syringe preparation station 130 through tubing and valves such as are shown in FIG. 1.

In another embodiment, the ozone generator 450 includes a solid electrolyte oxygen separator (SEOS) to generate substantially pure oxygen on demand from the ambient air. In another embodiment, the ozone generator 450 includes a polymeric membrane electrolyte to generate substantially pure oxygen on demand from the ambient air. In another embodiment, the ozone generator 450 includes a pressure swing absorption (PSA) oxygen separator to generate substantially pure oxygen on demand from the ambient air. Other embodiments may implement a combination of two or more conventional ozone generation technologies.

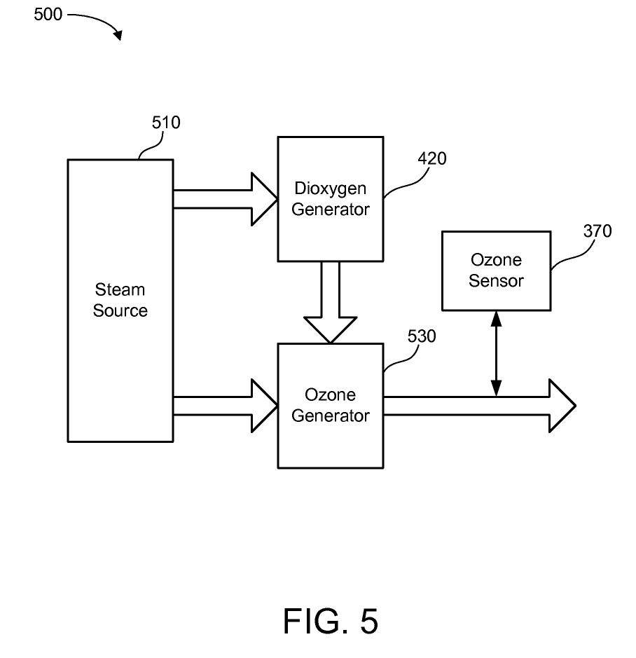

FIG. 5 depicts one embodiment of a process flow diagram 500 for generating ozone from steam in the syringe dispenser 100 of FIG. 2. In the illustrated embodiment, the syringe dispenser 100 includes a steam source 510, a dioxygen generator 420, an ozone generator 530, and an ozone sensor 370. The steam source supplies steam, water vapor, or another form of H₂O to the dioxygen generator 420 and/or the ozone generator 530. The dioxygen generator 420 generates dioxygen from the air content in the steam. The ozone generator 530 then generates ozone from the dioxygen generated by the dioxygen generator 420. The ozone generator 530 may include one or more of the technologies described above to generate the ozone from the dioxygen.

Additionally, the ozone generator 530 may generate ozone directly from the water content in the steam using, for example, the electrochemical ozone generator 360 described above. Thus, the ozone generator 530 may generate ozone from multiple oxygen sources using a combination of ozone generator technologies. In other embodiments, the ozone generator 530 may selectively generate ozone from one or more oxygen sources. Also, a condenser (not shown) may be included to facilitate the separation of the air and water components of the steam. The ozone sensor 370 monitors the generated ozone, and the ozone generator 530 sends the generated ozone to the syringe preparation station 130, as described above.

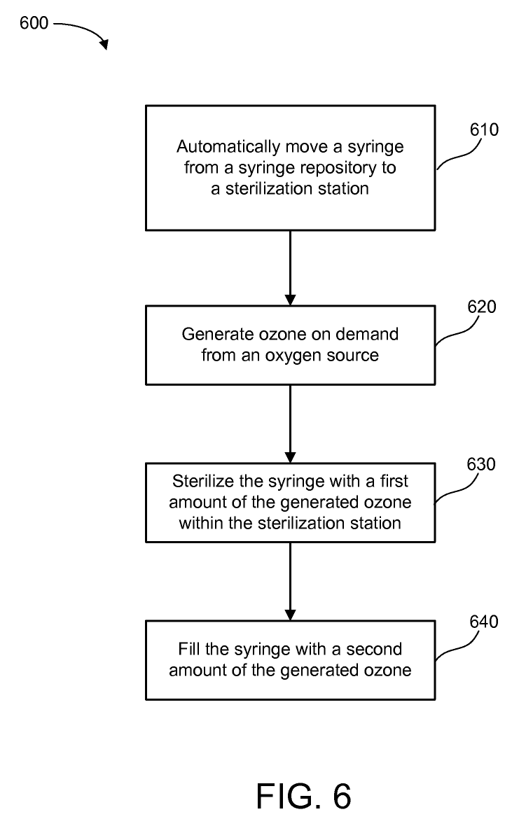

FIG. 6 depicts a flow chart diagram of an embodiment of a method 600 for autonomously sterilizing and filling a syringe 110. The illustrated method 600 includes automatically moving 610 the syringe 110 from the syringe repository 225 to a sterilization station (within the syringe preparation station 130) and generating 620 ozone on demand from an oxygen source. The method 600 also includes sterilizing 630 the syringe 110 with a first amount of the generated ozone within the sterilization station and filling 640 the syringe 110 with a second amount of the generated ozone.

Other embodiments of the method 600 may include fewer or more operations. For example, the method 600 also may include dispensing the sterilized, filled syringe 110 to a user. The method 600 also may include extracting oxygen from ambient air, for example, by moving the air to a solid electrolyte oxygen separator (SEOS), a polymeric membrane electrolyte oxygen separator, and/or a pressure swing absorption (PSA) oxygen separator to generate substantially pure oxygen. Other embodiments of the method 600 also include generating an amount of dioxygen from the ambient air and generating the ozone from the dioxygen. Some example technologies which may be implemented to generate the ozone include a corona discharge electrode, a UV light, a plasma generator, and an electrochemical ozone generator.

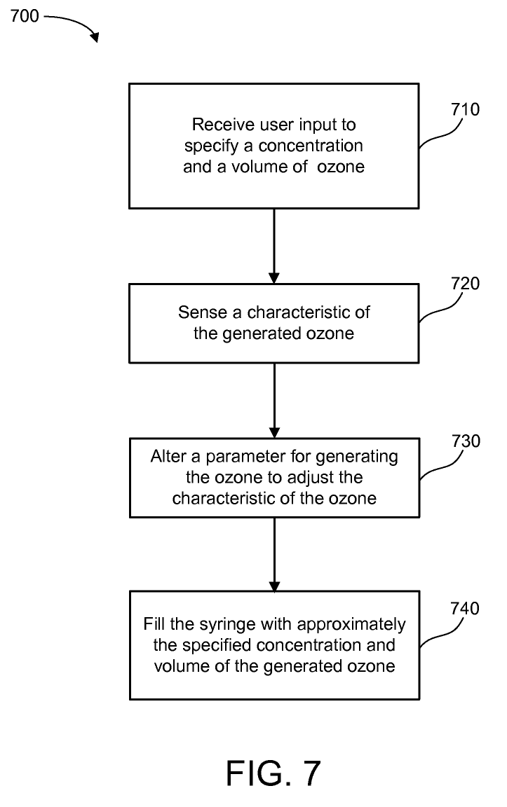

FIG. 7 depicts a flow chart diagram of an embodiment of a method 700 for generating ozone according to user input. The illustrated method 700 includes receiving 710 user input to specify a concentration and volume of the second amount of the generated ozone to be introduced into the syringe 110. The method 700 also includes sensing 720 a characteristic of the generated ozone and altering 730 a parameter for generating the ozone to adjust the characteristic of the generated ozone. The method 700 also includes filling 740 the syringe 110 with approximately the specified concentration and volume of the second amount of the generated ozone.

Other embodiments of the method 700 may include fewer or more operations. For example, the method 700 also may include communicating one or more control signals between the ozone generator 120, the controller 220, and the ozone sensor 370. In one embodiment, the controller 220 may process the signal from the ozone sensor 370 and control the generation of the ozone at the ozone generator 120. The controller 220, the ozone sensor 370, and the communication channels within the syringe dispenser 100 are not limited to simply electrical implementations, but also may be include mechanical or electromechanical implementations.

The method 700 for generating ozone according to the user input also may include receiving excess ozone from the syringe 110 and disposing of the excess ozone in the scrubber 170. Similarly, the method 700 may also include evacuating excess ozone from the sterilization station within the syringe preparation station 130 and disposing of the excess ozone in the scrubber 170.

While many embodiments are described herein, at least some embodiments present a technical application with certain advantages over conventional technologies. As one example, a therapeutic administrator may use a tabletop implementation of the syringe dispenser 100 to dispense sterilized, filled syringes 110 on demand. The syringe dispenser 100 dispenses the syringe 110 in accordance with specified parameters such as ozone volume and concentration inputs for a patient. The syringe dispenser 100 automatically moves a syringe 110 from the syringe repository 225 and sterilizes the syringe 100 with ozone generate on demand from the ambient air, tap water, or another oxygen source available onsite. The syringe 110 is also filled with ozone generated on demand having the precise characteristics meeting the specified parameters. The syringe dispenser may vend the sterile and filled syringe 110 into a dispensary for on-demand access to the ozone-filled syringe 110.

Some advantages of at least one embodiment of the syringe dispenser 100 include generating the therapeutic agent ozone on demand. Generating ozone on demand avoids aspects of inventory control to accurately account for the shelf-life of stored perishable therapeutic and medicinal agents. Since the syringe dispenser 100 is a self-contained apparatus, there is no contamination risk, or the contamination risk is minimized, associated with servicing and refilling syringes 110 and agents in the syringe dispenser 100. Also, according to an embodiment, the syringe dispenser 100 may use commonly available and inexpensive raw materials such as ambient air and tap water.

Another advantage of at least one embodiment of the syringe dispenser 100 is that the syringe dispenser 100 is easily portable and may sit on a desktop or on the top of a laboratory bench in a medical facility, a therapy clinic, or a veterinary doctor's office. Because of its ease of portability and self-containment, the syringe dispenser 100 may be used on-site in hazardous and hostile environments to service, for example, construction and military personnel. The syringe dispenser 100 may also be easily leased or sold to treatment facilities. Alternatively, the syringe dispenser may be provided free-of-charge, with payments made for each unit dispensed from the syringe dispenser 100.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A method for autonomously sterilizing and filling a syringe, the method comprising:
automatically moving the syringe from a syringe repository to a sterilization station;
generating ozone on demand from an oxygen source;
sterilizing the syringe with a first amount of the generated ozone within the sterilization station; and
filling the syringe with a second amount of the generated ozone.

2. The method of claim 1, further comprising dispensing the sterilized, filled syringe to a user.

3. The method of claim 1, wherein generating the ozone on demand from the oxygen source further comprises extracting oxygen from ambient air.

4. The method of claim 3, wherein extracting the oxygen from the ambient air further comprises moving the ambient air to a solid electrolyte oxygen separator (SEOS) to generate substantially pure oxygen.

5. The method of claim 3, wherein extracting the oxygen from the ambient air further comprises moving the ambient air to a polymeric membrane electrolyte oxygen separator to generate substantially pure oxygen.

6. The method of claim 3, wherein extracting the oxygen from the ambient air further comprises moving the ambient air to a pressure swing absorption (PSA) oxygen separator to generate substantially pure oxygen.

7. The method of claim 3, wherein generating the ozone on demand further comprises generating an amount of dioxygen from the ambient air and generating the ozone from the dioxygen.

8. The method of claim 7, wherein generating the ozone further comprises generating the ozone by passing the dioxygen near a corona discharge electrode.

9. The method of claim 7, wherein generating the ozone further comprises generating the ozone by exposing the dioxygen to an ultraviolet (UV) light.

10. The method of claim 1, wherein generating the ozone on demand from the oxygen source further comprises electrochemically generating the ozone from water.

11. The method of claim 10, wherein the water comprises sterile deionized water.

12. The method of claim 11, further comprising generating the sterile deionized water from tap water.

13. The method of claim 1, further comprising:
sensing a characteristic of the generated ozone; and
altering a parameter for generating the ozone to adjust the characteristic of the generated ozone.

14. The method of claim 1, further comprising:
receiving a user input to specify a volume of the second amount of the generated ozone to be introduced into the syringe; and
filling the syringe with approximately the specified volume of the second amount of the generated ozone.

15. The method of claim 14, further comprising generating the ozone with a specified concentration, wherein the user input specifies the concentration.

16. The method of claim 1, further comprising receiving excess ozone from the syringe and disposing of the excess ozone.

17. The method of claim 1, further comprising evacuating excess ozone from the sterilization station and disposing of the excess ozone.

18. The method of claim 1, further comprising disposing of excess ozone by directing the excess ozone to a scrubber.

19. The method of claim 1, wherein sterilizing the syringe further comprises sterilizing a used syringe.

20. An apparatus to deliver a sterile, filled syringe to a user, the apparatus comprising:
a controller configured to accept input from the user and to convert the input into at least one electrical control signal;
an ozone generator coupled to the controller, the ozone generator configured to generate ozone on demand according to the input from the user; and
a syringe preparation station coupled to the ozone generator, the syringe preparation station configured to sterilize the syringe with a first amount of the ozone and to fill the syringe with a second amount of the ozone.

21. The apparatus of claim 20, further comprising a syringe dispenser coupled to the syringe preparation station, the syringe dispenser configured to dispense the sterile, filled syringe to the user.

22. The apparatus of claim 20, further comprising an air intake coupled to the ozone generator, the air intake configured to introduce ambient air into the ozone generator, wherein the ozone generator is further configured to generate the ozone on demand from the ambient air according to the input from the user.

23. The apparatus of claim 20, wherein the ozone generator further comprises a solid electrolyte oxygen separator (SEOS) configured to generate substantially pure oxygen on demand from the ambient air.

24. The apparatus of claim 20, wherein the ozone generator further comprises a polymeric membrane electrolyte oxygen separator configured to generate substantially pure oxygen on demand from the ambient air.

25. The apparatus of claim 20, wherein the ozone generator further comprises a pressure swing absorption (PSA) oxygen separator configured to generate substantially pure oxygen on demand from the ambient air.

26. The apparatus of claim 20, wherein the ozone generator further comprises a dioxygen generator coupled to an ambient air source, the dioxygen generator configured to generate dioxygen on demand from the ambient air.

27. The apparatus of claim 26, further comprising a corona discharge generator coupled to the dioxygen generator, the corona discharge generator configured to generate ozone on demand from the dioxygen.

28. The apparatus of claim 26, further comprising an ultraviolet (UV) light coupled to the dioxygen generator, the UV light configured to generate ozone on demand from the dioxygen.

29. The apparatus of claim 20, further comprising a water intake coupled to the ozone generator, the water intake configured to introduce water into the ozone generator, wherein the ozone generator is further configured to generate the ozone on demand from the water according to the input from the user.

30. The apparatus of claim 29, wherein the water comprises sterile deionized water.

31. The apparatus of claim 29, further comprising a deionized water generator coupled to the water intake, the deionized water generator configured to generate sterile deionized water from the water according to the input from the user.

32. The apparatus of claim 29, further comprising a water reservoir coupled to the water intake, the water reservoir configured to hold the water for the ozone generator.

33. The apparatus of claim 32, further comprising a water treatment module coupled between the water reservoir and the ozone generator, the water treatment module configured to treat the water for the ozone generator.

34. The apparatus of claim 20, further comprising an ozone sensor coupled to the ozone generator and the controller, the ozone sensor configured to sense a characteristic of the generated ozone and to generate an ozone sensor signal indicative of the sensed characteristic of the generated ozone.

35. The apparatus of claim 20, wherein the ozone generator is configured to generate ozone with a specified concentration, wherein the user input specifies the concentration.

36. The apparatus of claim 20, wherein the ozone generator is configured to generate ozone with a specified volume, wherein the user input specifies the volume.

37. The apparatus of claim 20, further comprising a pump coupled to the syringe preparation station, the pump configured to evacuate excess ozone from a sterilization station of the syringe preparation station and to dispose of the excess ozone.

38. The apparatus of claim 37, further comprising a scrubber coupled to the pump, the scrubber configured to receive the excess ozone from the sterilization station and to dispose of the excess ozone.

39. The apparatus of claim 20, further comprising a syringe repository coupled to the syringe preparation station, the syringe repository configured to hold a plurality of unfilled syringes.

40. An apparatus to accept input from a user and to deliver a sterile, filled syringe to the user, comprising:
means for automatically moving the syringe from a syringe repository to a sterilization station;
means for generating ozone on demand from an oxygen source;
means for sterilizing the syringe with a first amount of the generated ozone within the sterilization station; and
means for filling the syringe with a second amount of the generated ozone.

41. The apparatus of claim 40, wherein the oxygen source comprises an air source, a water source, or a steam source.

42. The apparatus of claim 40, further comprising a means for dispensing the sterile, filled syringe to the user.

43. An apparatus to deliver a sterile, filled syringe to a user, the apparatus comprising:
a controller configured to accept input from the user and to convert the input into at least one electrical signal;
an ozone generator coupled to the controller, the ozone generator to generate ozone on demand according to the input from the user;
an ozone sensor coupled to the ozone generator and to the controller, the sensor configured to sense a characteristic of the ozone;
a syringe preparation station coupled to the ozone generator, the syringe preparation station configured to sterilize the syringe with a first amount of ozone and to fill the syringe with a second amount of ozone;
a scrubber coupled to the syringe preparation station and the ozone generator, the scrubber configured to receive excess ozone from the ozone generator and dispose of the excess ozone.

44. The apparatus of claim 43, further comprising a pump coupled to the ozone generator and the syringe preparation station, the pump configured to move ozone.

45. The apparatus of claim 43, further comprising a syringe repository coupled to the syringe preparation device, the syringe repository configured to hold unfilled syringes.

46. The apparatus of claim 43, further comprising an interface module coupled to the ozone generator, the interface module configured to accept air, water, or steam for the ozone generator and to return to the user a sterile filled syringe from the syringe preparation station.

47. The apparatus of claim 46, further comprising an air mover coupled to the interface module, the air mover configured to move air or steam from a source outside the ozone generator into the ozone generator.

48. The apparatus of claim 46, wherein the interface module is configured to accept a used syringe from the user.

49. An apparatus to accept a syringe from a user and to deliver a sterile, filled syringe to the user, comprising:
means for accepting and delivering a syringe from and to the user;
means for automatically moving the syringe within the apparatus;
means for generating ozone on demand from an oxygen source;
means for sterilizing the syringe with a first amount of the generated ozone within a sterilization station; and
means for filling the syringe with a second amount of the generated ozone.

50. The apparatus of claim 49, further comprising a means for accepting water or air or any combination thereof into the oxygen source from outside the apparatus.

51. The apparatus of claim 49, further comprising a means for controlling the ozone generator based on an at least one parameter input from the user for adjusting at least one characteristic of the generated ozone.

52. The apparatus of claim 49, further comprising a means for storing a plurality of the syringes after sterilization but before fill, and for enabling disposing of a store of used syringes.
